(19) United States
(12) Patent Application Publication
(10) Pub. No.: US 2012/0058185 A1
(43) Pub. Date: Mar. 8, 2012

(54) STABLE PHARMACEUTICAL COMPOSITIONS OF OLANZAPINE AND PROCESS FOR THEIR PREPARATION

(76) Inventors: Pattanayak Durgaprasad, Maharashtra (IN); Tallam Satyanarayana, Maharashtra (IN); V Satyanarayana, Maharashtra (IN); Rampal Ashok, Haryana (IN)

(21) Appl. No.: 13/321,366

(22) PCT Filed: May 20, 2010
(86) PCT No.: PCT/IN10/00324

§ 371 Date: Nov. 18, 2011

(30) Foreign Application Priority Data
May 22, 2009 (IN) 1282/MUM/2009

(51) Int. Cl. A61K 9/36; A61K 9/30; A61P 25/18; A61K 31/551

(52) U.S. Cl. 424/479; 514/220; 424/475

(57) ABSTRACT

The present invention provides a stable pharmaceutical composition of Olanzapine or its pharmaceutically acceptable salt comprising coated tablet formulations of Olanzapine or its pharmaceutically acceptable salt, wherein the coating consists essentially of pectin or copovidone, and a process of making the same.

### FIELD OF THE INVENTION

The present invention relates to stable pharmaceutical compositions of Olanzapine or its pharmaceutically acceptable salt and process for their preparation.

### BACKGROUND OF THE INVENTION

Olanzapine, 2-methyl-4-(4-methyl-1-piperazinyl)-10H-thieno[2,3-b][1,5]benzodiazepine, is an atypical antipsychotic, belonging to the class of drugs known as thienobenzodiazepines. It is available in the United States of America as ZYPREXA® Tablets, ZYPREXA® ZYDIS® Orally Disintegrating Tablets and ZYPREXA® IntraMuscular Olanzapine for Injection. Olanzapine is indicated in the United States of America for the treatment of schizophrenia, bipolar disorder and agitation associated with schizophrenia and bipolar I mania.

Olanzapine is disclosed per se in U.S. Pat. No. 5,229,382. The intrinsic nature of Olanzapine causes formulation problems such as moisture, sensitivity, propensity discoloration, metastability of various crystalline and amorphous forms and degradation after compounding into tablets.

U.S. Pat. No. 5,919,485 relates to a solid oral formulation comprising Olanzapine, wherein the formulation is coated with a polymer selected from hydroxypropyl methyl cellulose, hydroxyethyl cellulose, methyl hydroxyethyl sodium carboxymethyl cellulose, hydroxypropyl cellulose, polyvinylpyrrolidone, dimethylaminoethyl methacrylate, methyl acrylate acid ester copolymer, ethyl acrylate-methyl methacrylate copolymer, methyl cellulose, and ethyl cellulose.

U.S. Pat. No. 6,190,698 relates to a solid oral formulation consisting essentially of Olanzapine as an active ingredient intimately mixed with lactose, hydroxypropyl cellulose, crospovidone, microcrystalline cellulose, and magnesium stearate; wherein said solid oral formulation is coated with hydroxypropyl methyl cellulose, said hydroxypropyl methyl cellulose is further coated with an aqueous dispersion film coat.

U.S. Pat. No. 6,780,433 relates to a method for preparing a stable pharmaceutically elegant solid oral formulation of Olanzapine having a polymer coating selected from the group consisting of hydroxypropyl methyl cellulose, hydroxyethyl cellulose, methylhydroxyethylcellulose, sodium carboxymethylcellulose, hydroxypropylcellulose, polyvinyl pyrrolidone, dimethylaminoethyl methacrylatemethylacrylate acid ester copolymer, ethyl acrylate-methylacrylate copolymer, methylcellulose, and ethylcellulose, wherein the polymer coating is free of polyethylene glycol, comprised of using a high shear aqueous wet granulation with fluid bed drying.

U.S. Pat. No. 7,229,643 relate:; to a solid oral formulation comprising Olanzapine wherein the solid oral formulation is coated with a polymer which is hydroxypropyl methylcellulose wherein the polymer coating is free of polyethylene glycol.

United States Patent Application No. 20010020032 discloses solid oral formulation comprising Olanzapine as an active ingredient with one or more pharmaceutically acceptable excipients, wherein the Olanzapine is coated with a polymer selected from the group consisting of cetyl alcohol, cetyl esters wax, carnauba wax, shellac, beeswax, magnesium stearate, hydroxypropyl methyl cellulose, hydroxyethyl cellulose, methylhydroxyethylcellulose, sodium carboxymethylcellulose, hydroxypropylcellulose, polyvinyl pyrrolidone, dimethylaminoethyl methacrylatemethylacrylate acid ester copolymer, ethylacrylate-methylmethacrylate copolymer, methylcellulose, and ethylcellulose.

PCT Application No. 2007049304 discloses a stable solid oral pharmaceutical formulation comprising Olanzapine or a pharmaceutically acceptable salt thereof and pharmaceutically acceptable excipients, wherein said formulation is coated with stabilized coating employing a selective polymer selected from hydroxypropyl methyl cellulose phthalate, polyvinyl acetate phthalate, cellulose acetate phthalate, hydroxypropyl methyl cellulose acetate succinate, polyvinyl alcohol, vinyl acetate copolymer, pullulan gum or zein or a combination thereof. It is disclosed in the specification that the composition which is coated may be in the form of compressed tablets, moulded tablets, products prepared by extrusion and the likes. It is disclosed that it is preferred that the formulation is in a tablet form; however, granule formulation may be used as well.

The oral formulations disclosed in the prior art demand further improvements in light of the moisture sensitive and metastable nature of Olanzapine. Also, oral formulations are yet desired to provide improved stability of Olanzapine in tablet formulations in light of the above-mentioned problems associated with Olanzapine.

The present invention provides a stabilized formulation of Olanzapine, employing a selective polymer for coating, which overcomes the drawbacks of processes recited in prior arts.

It has been surprisingly found that a stable pharmaceutical composition of Olanzapine or its pharmaceutically acceptable salt, can be obtained by coating the Olanzapine tablet formulations with pectin or copovidone, and optionally one or more pharmaceutically acceptable excipients.

### OBJECT OF THE INVENTION

It is an object of the present invention to overcome or ameliorate at least one of the disadvantages of the prior art in making a stable pharmaceutical composition of Olanzapine or a pharmaceutically acceptable salt thereof.

It is an object of the present invention to provide pharmaceutical compositions of Olanzapine or its pharmaceutically acceptable salt and process for their preparation.

At least one of the preceding objects is met, in whole or in part, by a stable pharmaceutical composition of Olanzapine or its pharmaceutically acceptable salt comprising coated pharmaceutical composition of Olanzapine or its pharmaceutically acceptable salt, wherein the coating consists essentially of pectin or copovidione, and optionally one or more pharmaceutically acceptable excipients.

### SUMMARY OF THE INVENTION

The present invention provides a stable pharmaceutical composition of Olanzapine or its pharmaceutically acceptable salt and a process for its preparation.

The intrinsic nature of Olanzapine causes formulation problems such as moisture sensitivity, propensity for discoloration, metastability of various crystalline and amorphous forms and degradation of the drug after compounding into tablets. The present invention provides a stabilized tablet formulation of Olanzapine, employing a selective polymer for coating, which overcomes the drawbacks of processes recited in prior arts. We have found that a stable pharmaceutical composition of Olanzapine or its pharmaceutically acceptable salt, can be obtained by coating the Olanzapine tablet formulations with pectin or copovidone, and optionally one or more pharmaceutically acceptable excipients.

The invention may be summarized as given below:

A) A stable pharmaceutical composition of Olanzapine or its pharmaceutically acceptable salt comprising coated tablet formulations of Olanzapine or its pharmaceutically acceptable salt, wherein the coating consists essentially of pectin or copovidone, and optionally one or more pharmaceutically acceptable excipients.

B) A stable composition as in A above, wherein the pectin is used in amounts ranging from about 0.05% w/w to about 0.5% w/w.

C) A stable composition as in A above, wherein the copovidone is used in amounts ranging from about 0.5% w/w to about 5% w/w.

D) A process for preparing a stable pharmaceutical composition or Olanzapine or its pharmaceutically acceptable salt comprising coating the tablet formulations of Olanzapine or its pharmaceutically acceptable salt with a coating consisting essentially of pectin or copovidone, and optionally one or more pharmaceutically acceptable excipients.

### DETAILED DESCRIPTION OF THE INVENTION

Before the present process and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the step” includes reference to one or more step and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “stable” as used herein refers to chemical stability of Olanzapine in solid dosage forms wherein there is no change in assay values and dissolution and/or the total impurity remains less than 1%, when the dosage form is kept at 40° C./75% RH for 3 months.

The present invention relates to a stable pharmaceutical composition of Olanzapine or its pharmaceutically acceptable salt comprising coated tablet formulations of Olanzapine or its pharmaceutically acceptable salt, wherein the coating consists essentially of pectin or copovidone, and optionally one or more pharmaceutically acceptable excipients.

The present invention provides a process for preparing a stable pharmaceutical composition of Olanzapine or its pharmaceutically acceptable salt comprising coating the tablet formulations of Olanzapine or its pharmaceutically acceptable salt with a coating consisting essentially of pectin or copovidone, and optionally one or more pharmaceutically acceptable excipients.

Olanzapine is effective over a wide dosage range, the actual dose administered depending on the condition treated. For example, in the treatment of adult humans, dosages of from 0.25 to 50 mg, preferably 1 to 30 mg, and most preferably 1 to 20 mg per day may be used. A once-daily dosage is normally sufficient, although divided doses may be administered. A preferred composition of the invention comprises 0.25 to 50 mg, more preferably, 1 to 30 mg and even more preferably, 1 to 20 mg of Olanzapine (calculated as the free anhydrous base). Preferably, the Olanzapine as used herein is in the free form.

In the present invention, is provided a process for preparing a stable pharmaceutical composition of Olanzapine having polymer based film coatings, particularly by using pectin or copovidone as film forming agents.

Pectins are anionic polyelectrolytes having a linear chain or (1-4) linked alpha D-galactopyranosyluronic acid units that forms the pectin-backbone, a homogalacturonan. Thus, pectins are polygalactouronic acid and the chain molecules are negatively charged at neutral pH. Copovidone is a copolymer of 1-vinyl-2-pyrrolidone and vinyl acetate in the mass proportion of 3:2. Both pectin and copovidone are dissolved in water and most pharmaceutically acceptable solvents, providing coating properties such as less hygroscopic and improved film toughness with better film adhesion, increased coating application rates and gloss, preventing fading and color shifts & thereby imparting stability to the pharmaceutical compositions. A relatively low content of pectin ranging from about 0.05% w/w to about 0.5% w/w and of copovidone from about 0.5% w/w to about 5% w/w, in the present stable pharmaceutical composition of Olanzapine or its pharmaceutically acceptable salt, is sufficient to obtain tablet films with improved stability properties.

The pharmaceutical composition of the present invention may further comprise conventional pharmaceutically acceptable excipients. Conventional pharmaceutical excipients include those which function in a dosage form, for example, as a disintegrant, lubricant, glidant, fillers or diluents, wicking agents and the like.

The fillers or diluents that may be used in the stable oral pharmaceutical composition of the present invention include microcrystalline cellulose, mannitol, dextrates, dextrins, dextrose, fructose, lactose, lactitol, maltitol, maltodextrins, maltose and the like and mixtures thereof. Preferred filler is microcrystalline cellulose or lactose or a combination thereof. The diluents or fillers may typically be used in amounts ranging from about 0.1% w/w to about 80% w/w.

The disintegrants that may be used in pharmaceutical composition of the present invention include carboxymethylcellulose calcium, carboxymethylcellulose sodium, colloidal silicon dioxide, croscarmellose sodium, crospovidone, guar gum, magnesium aluminium silicate, methylcellulose, microcrystalline cellulose, polacrilin potassium, powdered cellulose, pregelatinized starch, sodium alginate, sodium starch glycolate, and starch and the like or mixtures thereof. The disintegrants may typically be used in amounts ranging from about 0.1% w/w to about 50% w/w.

Examples of wicking agents that may be used in the present invention include colloidal silicon dioxide, kaolin, titanium dioxide, fumed silicon dioxide, m-pyrol, vinylpyrrolidone polymers such as povidone, or crosslinked polyvinylpyrrolidone such as crospovidone; cellulose and cellulose derivatives such as microcrystalline cellulose, methylcellulose, ethylcellulose, hydroxypropylcellulose, hydroxypropyl cellulose, carboxyalicyl celluloses and their alkali salts; sodium starch glycolate, starch and starch derivatives, ion-exchange resins and the like and mixtures thereof.

The binders used in the present invention may be selected from the group comprising of starch, gelatin, dextrin, maltodextrin, natural and synthetic gums like acacia, alginic acid, sodium alginate, guar gum, extract of fish moss, ghatti gum, mucilage of isapol husks, carboxymethylcellulose, methylcellulose, hydroxyethyl cellulose, hydroxypropyl cellulose, hydroxypropyl methylcellulose, polyvinylpyrrolidone, veegum, arabogalactan and the like and mixtures thereof.

The lubricants used in the present invention may be selected from the group consisting, of talc, magnesium stearate, calcium stearate, stearic acid, hydrogenated vegetable oils, polyethylene glycol and the like and mixtures thereof. Generally the amount of the lubricants used in the present invention may typically vary from about 0.001% w/w to about 5% w/w of the composition.

The typical glidants that may be included in the present invention include colloidal silicon dioxide, talc and the like. The amounts of glidants used in the present invention may typically range from about 0.1% w/w to about 5% w/w of the composition.

The coloring agents include any suitable FDA approved colors for oral use.

The pharmaceutical compositions of the present invention may be prepared by the conventional processes such as wet granulation, dry granulation or direct compression. In wet granulation, Olanzapine or its pharmaceutically acceptable salt is mixed with suitable excipients and granulated, followed by screening and drying of the damp mass. The dried mass may be screened, lubricated and compressed. Dry granulation can be done by two processes: (1) slugging, which involves mixing the Olanzapine or its pharmaceutically acceptable salt with suitable excipients, slugging, dry screening, lubrication and compression, or (2) roller compaction process. Direct compression involves compressing tablets directly from the physical mixture of Olanzapine or its pharmaceutically acceptable salt and the suitable excipients. Alternatively the pharmaceutical compositions of the present invention may be obtained by preparing placebo granules comprising the suitable pharmaceutically acceptable excipients, and mixing these with Olanzapine to obtain a blend, which may be encapsulated or compressed into tablets. The tablet formulations of Olanzapine or its pharmaceutically acceptable salt obtained by above cited methods, are coated by the coating dispersion made by using pectin or copovidone. These methods provide compositions of Olanzapine that are stable.

The following examples are intended to illustrate the scope of the present invention in all its aspects but not to limit it thereto.

### EXAMPLE 1

The stable oral pharmaceutical composition comprising Olanzapine or its pharmaceutically acceptable salt may be prepared as given in Table 1.

TABLE 1

| | Quantity | |
|---|---|---|
| Name of Ingredients | (Mg/Tab) | % w/w |
| Core | | |
| Olanzapine | 5.00 | 2.45 |
| Lactose monohydrate | 157.50 | 77.21 |
| Hydroxypropyl cellulose | 6.50 | 3.19 |
| Crospovidone | 5.00 | 2.45 |
| Microcrystalline cellulose | 25.00 | 12.25 |
| Magnesium stearate | 1.00 | 0.49 |
| Purified water | q.s. | |
| Coating | | |
| Pectin | 0.137 | 0.067 |
| Titanium dioxide | 3.79 | 1.858 |
| Red Iron oxide | 0.0171 | 0.0083 |
| Triacetin | 0.0206 | 0.0100 |
| Talc | 0.0343 | 0.0167 |
| Acetone | q.s. | |
| Purified water | q.s. | |
| Total weight | 204.00 | 100.00 |

The stable pharmaceutical composition of Olanzapine was prepared by a process as follows:

Olanzapine, lactose monohydrate, crospovidone and hydroxypropyl cellulose were sifted through # 40 sieve, mixed geometrically and dry mixed for 10 min. This premix was then granulated in RMG using binder solution which was prepared by dissolving the hydroxypropyl cellulose in water. The obtained wet mass was then dried in FBD at 60° C. and screened through # 24 sieve and mixed with microcrystalline cellulose and/or crospovidone for 20 mm and lubricated with magnesium stearate which was previously passed through # 60 sieve, for 5 min. This blend was then compressed into tablets using appropriate tooling. The tablets were coated with the coating dispersion made by using pectin.

### EXAMPLE 2

The stable oral pharmaceutical composition comprising Olanzapine or its pharmaceutically acceptable salt may be prepared as given in Table 2.

TABLE 2

| | Quantity | |
|---|---|---|
| Name of Ingredients | (Mg/Tab) | % w/w |
| Core | | |
| Olanzapine | 5.00 | 2.381 |
| Lactose monohydrate | 156.00 | 74.286 |
| Hydroxypropyl cellulose | 8.00 | 3.81 |
| Crospovidone | 10.00 | 4.762 |
| Microcrystalline cellulose | 20.00 | 9.524 |
| Magnesium stearate | 1.00 | 0.476 |
| Purified water | q.s. | |
| Coating | | |
| Pectin | 0.343 | 0.163 |
| Titanium dioxide | 9.475 | 4.512 |
| Red Iron oxide | 0.043 | 0.020 |
| Triacetin | 0.052 | 0.025 |
| Talc | 0.087 | 0.041 |
| Acetone | q.s. | |
| Purified water | q.s. | |
| Total weight | 210.00 | 100.00 |

The composition was prepared as per the procedure given in example 1.

### EXAMPLE 3

The stable oral pharmaceutical composition comprising Olanzapine or its pharmaceutically acceptable salt may be prepared as given in Table 3.

TABLE 3

| | Quantity | |
|---|---|---|
| Name of ingredients | (Mg/Tab) | % w/w |
| Core | | |
| Olanzapine | 5.00 | 2.45 |
| Lactose monohydrate | 157.50 | 77.21 |
| Hydroxypropyl cellulose | 6.50 | 3.19 |
| Crospovidone | 5.00 | 2.45 |
| Microcrystalline cellulose | 25.00 | 12.25 |
| Magnesium stearate | 1.00 | 0.49 |
| Purified water | q.s. | |
| Coating | | |
| Copovidone | 2.0 | 0.980 |
| Titanium dioxide | 1.6 | 0.784 |
| Red Iron oxide | 0.0171 | 0.0084 |
| Triacetin | 0.20 | 0.0980 |
| Talc | 0.1829 | 0.0896 |
| Purified water | q.s. | |
| Total weight | 204.00 | 100.00 |

The composition was prepared as per the procedure given in example 1 except that the tablets were coated with the coating dispersion made by using copovidone instead of pectin.

### EXAMPLE 4

The stable oral pharmaceutical composition comprising Olanzapine or its pharmaceutically acceptable salt may be prepared as given in Table 4.

TABLE 4

| | | | |
|---|---|---|---|
| | | Qty mg/ | |
| Sr. No | Ingredient | Tablet | % w/w |

CORE

| | | | |
|---|---|---|---|
| 1. | Olanzapine | 5.000 | 2.38 |
| 2. | Lactose monohydrate | 155.500 | 74.05 |
| 3. | Low substituted Hydroxypropyl cellulose | 10.000 | 4.76 |
| 4. | Hydroxypropyl cellulose | 8.000 | 3.81 |
| 5. | Mannitol | 20.000 | 9.52 |
| 6. | Magnesium stearate | 1.500 | 0.71 |
| 7. | Purified water | q.s. | |

COATING

| | | | |
|---|---|---|---|
| 8. | Pectin | 0.350 | 0.17 |
| 9. | Triacetin | 0.070 | 0.03 |
| 10. | Titanium dioxide | 1.500 | 0.71 |
| 11. | Talc | 8.080 | 3.85 |
| 12. | Purified water | q.s. | |
| | Total | 210 | 100 |

The composition was prepared as per the procedure given in example 1.

### EXAMPLE 5

The composition prepared in example 4 was subjected to stability testing at 40° C./75% RH (Accelerated condition) The results are given below in table 5.

TABLE 5

| | | Related | |
|---|---|---|---|
| | | substances/ | |
| | Assay | Impurities | % Dissolution |
| Period | (in %) | (in %) | at 30 min |
| Initial | 100.67 | 0.199 | 97.6 |
| 1 Month | Not done | 0.282 | 99.3 |
| 2 Month | Not done | 0.313 | 96.4 |
| 3 Month | 101.52 | 0.696 | 97.6 |

Thus it can be seen that, the composition of the present invention on storage at 40° C./75% RH for 3 months shows no change in assay values and dissolution and the total impurity content of the composition remains less than 1%.

Although the invention has been described in terms of particular embodiments and applications, one of ordinary skill in the art, in light of this teaching, can generate additional embodiments and modifications without departing from the spirit of or exceeding the scope of the claimed invention. It should be emphasized that the above-described embodiments of the present invention, particularly any “preferred” embodiments, are merely possible examples of the invention of implementations, merely set forth for a clear understanding of the principles of the invention. Accordingly, it is to be understood that the drawings and descriptions herein are preferred by way of example to facilitate comprehension of the invention and should not be construed to limit the scope thereof.

1. A stable pharmaceutical composition of Olanzapine or its pharmaceutically acceptable salt comprising coated tablet formulations of Olanzapine or its pharmaceutically acceptable salt, wherein the coating consists essentially of pectin or copovidone, and optionally one or more pharmaceutically acceptable excipients.

2. A stable composition as in claim 1, wherein the pectin is used in amounts ranging from about 0.05% w/w to about 0.5% w/w.

3. A stable composition as in claim 1, wherein the copovidone is used in amounts ranging from about 0.5% w/w to about 5% w/w.

4. A process for preparing a stable pharmaceutical composition of Olanzapine or its pharmaceutically acceptable salt comprising coating the tablet formulations of Olanzapine or its pharmaceutically acceptable salt with a coating consisting essentially of pectin or copovidone, and optionally one or more pharmaceutically acceptable excipients.
